(19) United States
(12) Patent Application Publication
(10) Pub. No.: US 2023/0137997 A1
(43) Pub. Date: May 4, 2023

(54) TOPICAL APPLICATOR

(71) Applicant: Robin Bisio Tripaldi, Alamo, CA (US)

(72) Inventor: Robin Bisio Tripaldi, Alamo, CA (US)

(21) Appl. No.: 17/978,867

(22) Filed: Nov. 1, 2022

Related U.S. Application Data
(60) Provisional application No. 63/274,456, filed on Nov. 1, 2021.

(51) Int. Cl. A61M 35/00; A61K 9/00

(52) U.S. Cl. CPC A61M 35/003; A61K 9/0014; A61M 2209/06

(57) ABSTRACT

A topical applicator has a cup filled with a desired medium, such as an analgesic, and is sealed with a peel-off lid. The cup has a lip region wherein an outer annular region is removably attached to the lid, and the inner annular region contains an adhesive to removably attach the applicator to skin in order to treat a specified area with the medium contained in the cup.

### CROSS-REFERENCE TO RELATED APPLICATIONS

This application claims the benefit of priority to prior-filed and co-pending U.S. Provisional Patent Application No. 63/274,456, filed Nov. 1, 2021, the complete contents of which is hereby incorporated herein by reference.

### BACKGROUND

### Technical Field

The present device relates to the field of topical gel application and more specifically to specialized, individual-use topical content applicators.

### Background

Individuals who have undergone medical treatment which requires the installation of a medical port in their person, are familiar with the procedures required to prepare for treatments which require a medical professional to access the port. Often accessing a port can be uncomfortable or painful and as such patients are frequently advised to apply lidocaine topically to the port and the surrounding region prior to arriving for the procedure requiring port access. However, topical lidocaine becomes highly fluid when it comes in contact with the body. Thus, it is not unusual for patients to have the lidocaine run down their bodies to undesired locations or simply not stay in place long enough to affect topical numbing of the skin. The common solution employed and recommended by medical professionals is to wrap the area in cellophane or plastic wrap in an effort to keep the lidocaine in place. However, those that have tried this are familiar with the fact that wrapping the area of the port is not a practical solution as the area where the port is installed may not be easily wrapped and/or the cellophane/plastic wrap simply does not adhere to the area sufficiently to keep the lidocaine in place. This same issue is true with many topical anesthetics and other topical analgesics which are not readily or immediately absorbed into the skin and must sit on the surface of the skin for a period of time to be effective.

What is needed is an apparatus to securely position a topical analgesic, anesthetic or other material against the skin such that the material is in contact with the skin for the desired period to have the desired effect.

### BRIEF DESCRIPTION OF THE DRAWINGS

Further details of the present device are explained with the help of the attached drawings in which:

FIG. 1 depicts a top view of the topical applicator of FIG. 1 in a closed position.

FIG. 2 depicts a top view of an open topical applicator.

FIG. 3 depicts an elevation view of the topical applicator of FIG. 1.

FIG. 4 depicts a top view of another embodiment of the present device.

FIG. 5 depicts an elevation view of the embodiment shown in FIG. 4.

### DETAILED DESCRIPTION

As used in the description herein and throughout the claims that follow, “a”, “an”, and “the” includes plural references unless the context clearly dictates otherwise. Also, as used in the description herein and throughout the claims that follow, the meaning of “in” includes “in” and “on” unless the context clearly dictates otherwise.

FIG. 1 depicts a top view of a closed topical applicator 100. As shown in FIG. 1, a topical applicator 100 can have a substantially circular geometry, but in other embodiments can have any other known and/or convenient configuration. In the embodiment depicted in FIG. 1, a topical applicator 100 is depicted in a closed or sealed position with a lid 110 covering the open end of a cup 102.

As shown in FIG. 1, a cup 102 can have a lip region 104. In the embodiment of FIG. 1, a lip region 104 can extend circumferentially outward from the edge of a cup 102, but in other embodiments can extend inward. In such embodiments, a lip region 104 can extend substantially perpendicularly from the edge of a cup 102. A lip region 104 can have a substantially flat, or rounded, or any other known and/or convenient configuration. In some embodiments, a cup 102 can be comprised of a polymer or any other known and/or convenient material, which can be biodegradable. In such embodiments, a cup 102 can be substantially rigid or flexible to any known and/or convenient degree.

As shown in FIG. 1, a lid 110 can be configured to substantially correspond with the dimensions of a cup 102. In some embodiments, a lid 110 can have at least one tab 112 that can extend outward past a lip region 104. As shown in FIG. 1, a tab 112 can have a rounded geometry, but in other embodiments can have a substantially rectangular shape or any other known and/or convenient geometry. In other embodiments, a lid 110 can in whole or in part extend past a lip region 104. In another alternative embodiment, a lip region 104 can have at least one notch to facilitate peeling off a lid 110. A lid 110 can be comprised of a polymer, metallic foil, or any other known and/or convenient material.

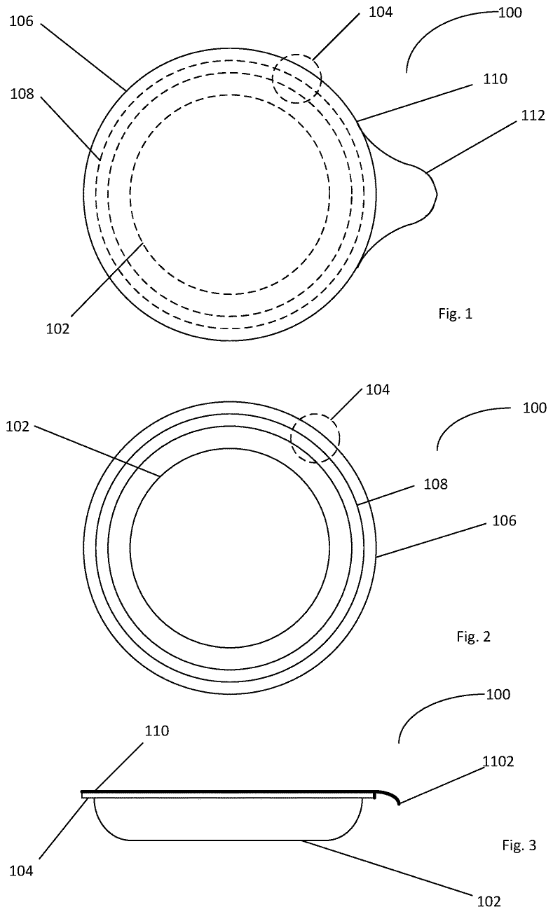

FIG. 2 depicts a top planar view of a cup 102 with a lid 110 removed. In the embodiment depicted in FIG. 1, a lip region 104 can be comprised of two annular regions 106 and 108, each having adhesives associated with each annular region 106 108. An exterior annual region 106 can have a first adhesive that is adapted and configured to selectively seal a lid 110 to a cup 102. In some embodiments, this first adhesive can contain an analgesic substance, such as, but not limited to lidocaine and/or any other known, convenient and/or desired substance contained within a cup 102 but allow selective removal of a lid 110 from a cup 102 when it is desired to expose the contents and opening of a cup 102 as well as an internal annular region 108 of a cup lid region 104. In the embodiment depicted in FIG. 2, an internal annular region 108 can be associated with an adhesive that is adapted and configured to selectively adhere to both a cup 102 and human or animal skin. In some embodiments, this second adhesive can contain an analgesic substance, such as, but not limited to lidocaine and/or any other known, convenient and/or desired substance contained within a cup 102.

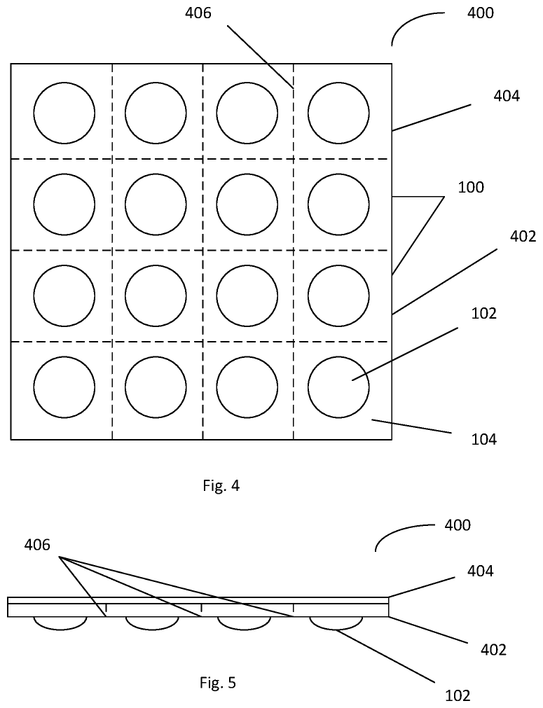

FIG. 3 depicts an elevation view of the topical applicator of FIG. 1. In the embodiment shown, a cup 102 can have a substantially rectangular cross-section with rounded edges at the bottom. In other embodiments, a cup 102 can have a substantially hemispherical or any other known and or convenient cross-sectional geometry.

In some embodiments, a cup 102 and a lip region 104 can be comprised of a flexible material or semi-rigid material such that it can conform to the shape of the body of the user to which the cup is attached. Moreover, in some embodiments a cup 102 can be comprised of a flexible material such that a cup 102 can be deformed by the user such that the media is held closer to the skin.

In some embodiments, a lid 110 can be a peel-off type lid 110 with a tab 112 to facilitate removal of a lid 110 from a cup 102. However, in alternate embodiments, a lid 110 can be any known, convenient and/or desired style or material and can selectively couple a cup 102 in any known, convenient and/or desired manner or via any known, convenient and/or desired mechanism, such as a screw-type mechanism. In some embodiments wherein a lid 110 is selectively coupled with a cup 102 other than via adhesive, adhesive may not be associated with an exterior annular region 106 and/or an exterior annual region 106 can comprise an additional annular application of skin adhesive.

In operation, a user can remove a lid 110 and expose both the interior of a cup 102, which can contain any known, convenient and/or desired topical application (and/or can be empty such that a user can introduce any desired media into a cup 102). A user can then (fill a cup 102 with desired media, if empty) and apply a lip region 104 of a cup 102 to the skin, thus adhering a cup 102 containing the media against the skin via a second adhesive in an internal annular region 108. In such embodiments, a topical applicator 100 can specifically target an area of skin contained with the circumference or perimeter of a topical applicator 100. In embodiments where a topical application is an analgesic, such as, but not limited to lidocaine, a topical applicator 100 can numb an area of skin contained with the circumference or perimeter of a topical applicator 100. When treatment is concluded, a user can then remove a topical applicator 100 from the area of skin on which it was deployed.

FIG. 4 depicts a top view of another embodiment of the present device. In such embodiments, topical applicators 100 can be produced in a sheet configuration to create a “pack” of multiple topical applicators 400. In such embodiments, individual topical applicators 100 can comprise an array in which topical applicators 100 can be removably connected. As shown in FIG. 4, instead of discreet, individual cups 102, a bottom sheet 402 can have an array of cups 102, which can be filled with a desired medium. The interstitial regions of a bottom sheet 402 can serve as lips 104 to cups 102. These lips 104 can comprise a pair of annular regions 106 108 having different adhesives, as discussed supra. A top sheet 404 can be comprised of foil, polymer, or any other known and/or convenient substantive. A top sheet 404 can be removably attached to a bottom sheet 402 via a first adhesive region 106.

As shown in FIG. 4, Substantially orthogonal perforations 406 can allow for individual topical applicators 100 to be broken off as needed. In such embodiments, the resulting corners of each topical applicator 100 can be free of adhesive to allow a user to easily peel off a top sheet 404 to open a topical applicator 100 and expose a cup 102. However, in other embodiments, a bottom sheet 402 can have cups 102 only partially connected tangentially. As described supra, this would also expose a second adhesive region 108 comprising an adhesive to removably attach a topical applicator 100 in direct contact with a desired skin surface. In other embodiments, a bottom sheet 402

FIG. 5 depicts an elevation view of the embodiment shown in FIG. 4. In such embodiments, a bottom sheet 402 can have a plurality of cups 102 protruding from the lower surface. Each cup 102 can be filled with a desired medium, such as, but not limited to a topical analgesic. A top sheet 404 can be removably attached to a bottom sheet 402 via a first adhesive region 106 surrounding the edge of each cup 102.

In use, a user can select an individual topical applicator 100 from a sheet 400 and remove it from the rest of a sheet 400 along perforations 406. The resulting individual applicator 100 can have a substantially rectangular lip surrounding the edge of a cup 102. A user can peel off a top sheet 404 to expose the contents of a cup 102 and a second adhesive 108. An individual applicator 100 can then be applied to a desired area of skin. When removal from the skin is desired, a user can use one of the adhesive-free corners to pull an applicator 100 off of the skin.

Although exemplary embodiments of the invention have been described in detail and in language specific to structural features and/or methodological acts above, it is to be understood that those skilled in the art will readily appreciate that many additional modifications are possible in the exemplary embodiments without materially departing from the novel teachings and advantages of the invention. Moreover, it is to be understood that the subject matter defined in the appended claims is not necessarily limited to the specific features or acts described above. Accordingly, these and all such modifications are intended to be included within the scope of this invention construed in breadth and scope in accordance with the appended claims.

What is claimed:

1. A topical applicator apparatus comprising:
a cup having an outwardly extended lip region, further comprising an outer annular region and an inner annular region.
said cup adapted to contain a desired medium;
a removable lid selectively coupled with said outer annular region of said cup via a first adhesive;
a second adhesive associated with said inner annular region.

2. The topical applicator of claim 1, wherein said first adhesive is adapted to selectively bond said lid and said outer annular region.

3. The topical applicator of claim 2, wherein said second adhesive is adapted to selectively bond said inner annular region and human or animal skin.

4. The topical applicator of claim 1, wherein said desired medium is an analgesic.

5. The topical applicator of claim 1, wherein said lid further comprises a tab extending outward past the lip region of said cup.

6. The topical applicator of claim 1, wherein said cup and said lid have a substantially circular geometry.

7. The topical applicator of claim 6, wherein said cup is comprised of a polymer.

8. The topical applicator of claim 7, wherein said lid is comprised of a metallic foil.

9. The topical applicator of claim 8, wherein said cup is deformable.

10. A topical applicator apparatus comprising:
a substantially circular cup having an outwardly extended lip region, further comprising an outer annular region and an inner annular region. said cup adapted to contain a desired analgesic;
a removable lid selectively coupled with said outer annular region of said cup via a first adhesive and further comprising a tab extending outward past said outwardly extended lip region;
a second adhesive adapted to bond said inner annular region and human or animal skin associated with said inner annular region.

11. A topical applicator apparatus comprising:
a bottom sheet having an upper surface and a lower surface with a plurality of protrusions on said lower surface forming cups with edges on said sheet;
wherein each cup is filled with a desired medium;
an outer annular region having a first adhesive and an inner annular region having a second adhesive;
a top sheet removably attached to the top surface of said bottom sheet via said outer adhesive;
a plurality of substantially orthogonal perforations through said top sheet and said bottom sheet delineating regions each containing one cup.

12. The topical applicator apparatus of claim 11, wherein said bottom sheet is comprised of a polymer.

13. The topical applicator apparatus of claim 12, wherein said top sheet is comprised of a metallic foil.
